(19) United States
(12) Patent Application Publication
(10) Pub. No.: US 2007/0163008 A1
(43) Pub. Date: Jul. 12, 2007

(54) Alfalfa plants with detectable tannin levels and methods for producing same

(75) Inventors: Lauren D. Johnson, Woodland, CA (US); David W. Johnson, West Salem, WI (US); Jay M. Sandman, West Salem, WI (US); Douglas K. Miller, West Salem, WI (US); Jonathan M. Reich, Woodland, CA (US)

Correspondence Address: COOLEY GODWARD KRONISH LLP;ATTN: Patent Group, Suite 500, 1200 - 19th Street, NW, WASHINGTON, DC, 20036-2402, US

(73) Assignee: Cal/West Seeds

(21) Appl. No.: 11/715,356

(22) Filed: Mar. 8, 2007

Related U.S. Application Data
(63) Continuation of application No. 10/698,426, filed on Nov. 3, 2003.
(60) Provisional application No. 60/422,859, filed on Nov. 1, 2002.

(51) Int. Cl. A01H 5/00; C12N 5/04

(52) U.S. Cl. 800/295; 435/419

(57) ABSTRACT

The invention provides alfalfa plants with detectable levels of tannins, especially condensed tannins, and methods of making alfalfa plants with detectable levels of tannins. Plants and plant parts of the invention are useful as forage for ruminants as they reduce the risk of bloat and reduce or eliminate the need for protein supplementation in ruminant diets.

### CROSS REFERENCE TO RELATED APPLICATION

This application is a continuation of U.S. application Ser. No. 10/698,426, filed Nov. 3, 2003, which claims priority to U.S. Provisional Patent Application No. 60/422,859, filed Nov. 1, 2002, each of which is herein incorporated by reference in its entirety.

### FIELD OF THE INVENTION

This invention relates to the field of alfalfa plants, and more specifically to improved alfalfa germplasm, such as improved alfalfa varieties, having increased tannin contents and methods for producing such germplasm.

### BACKGROUND OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

Alfalfa (Medicago sativa L.) is an important forage species for hay and pasture which has been referred to as the “Queen of the Forages” because of its high yields and feeding value. Alfalfa is recognized as the most widely adapted agronomic crop, as an effective source of biological nitrogen (N2) fixation, useful in the improvement of soil tilth, as an important source of protein yield/ha, and as an attractive source of nectar for honey bees. For a comprehensive review of the benefits of alfalfa as an agronomic crop, see Barnes et al., Highlights in the USA and Canada 1:2-24, In Alfalfa and Alfalfa Improvement, Hanson et al. (ed.), American Society of Agronomy, Monograph No. 29 (1988).

Although alfalfa originated in southwestern Asia, it is well adapted to a wide range of climates and soils in the United States. Alfalfa is classified into fall dormancy groups, numbered 1 to 10 which can be fitted into the plant hardiness zone map. Dormancy group 1 is very dormant and best suited for cold climates (such varieties would stop growing and go dormant over winter), and dormancy group 10 is very non-dormant and suited for very hot climates (such varieties would have high growth rates over a very long growing season and would have relatively high winter activity). For a comprehensive review of geographic adaptation of alfalfa, see Melton et al., Geographic Adaptation and Cultivar Selection 20: 595-620, In Alfalfa and Alfalfa Improvement, supra. Between 1900 and 1975 more than 160 cultivars were developed for production in North America. Most of the newer cultivars were selected for improved adaptation and multiple pest resistance. For a comprehensive review of the distribution, history and origin of alfalfa, see Michaud et al., World Distribution and Historical Development 2:25-91, In Alfalfa and Alfalfa Improvement, supra; and, Quiros et al., The Genus Medicago and the Origin of the Medicago sativa Complex 3:93-124, In Alfalfa and Alfalfa Improvement, supra.

The genus Medicago is widely distributed and comprises an array of diverse species that are either annual or perennial. The most recent taxonomic studies of the perennial species concluded that M. sativa is polymorphic. Lesins and Gillies (Taxonomy and cytogenetics of Medicago 353-386, In Alfalfa science and technology, C. H. Hanson (ed.), American Society of Agronomy, (1972)) defined the complex as M. sativa-falcata-glutinosa, and Gunn et al. (USDA Tech. Bull. No. 1574 (1978)) designated it as the M. sativa sensu lato complex.

M. sativa plants are autopolyploid organisms, or more specifically, autotetraploids. More specifically, M. sativa plants are polysomic polyploid organisms which display tetrasomic inheritance patterns.

Essentially all annual species are cleistogamous and are exclusively self-pollinated. Generally, the perennial species require tripping, as by insect visits to the floral structures, and will set seed from either self or cross-pollination. Crosses can be made among subspecies in the M. sativa complexes and between the cultivated tetraploids and wild diploids without special preparation of the parents. For a comprehensive review of the floral characteristics, plant culture, and methods of self-pollinating or hybridizing alfalfa, see D. K. Barnes, Alfalfa 9:177-187, In Hybridization of Crop Plants, Fehr et al. (ed.), American Society of Agronomy Inc. (1980).

Commercial alfalfa seed may be provided either in a synthetic variety or a hybrid variety. Commercial production of synthetic varieties may include a breeder seed production stage, a foundation seed production stage, a registered seed production stage and a certified seed production stage. Hybrid variety seed production may involve up to three stages including a breeder seed production stage, a foundation seed production stage and a certified seed production stage.

Efforts in developing healthy and productive alfalfa varieties often focus on breeding for disease and stress-resistant cultivars, for example, breeding for persistence, breeding for adaptation to specific environments, breeding for yield per se, and breeding for quality. Success has been attained in breeding for resistance to fungal, bacterial, insect, and nematode pests, including, but not limited to the development of varieties tolerant/resistant to bacterial wilt and common leaf spot (see, e.g., Elgin, Jr., et al., Breeding for Disease and Nematode Resistance 827-858, In Alfalfa and Alfalfa Improvement, supra) and to the spotted alfalfa aphid and alfalfa weevil (see, e.g., Sorensen et al., Breeding for Insect Resistance 859-902, In Alfalfa and Alfalfa Improvement, supra). Breeders have had less success in breeding for yield and quality per se (see, e.g., Hill et al., Breeding for Yield and Quality 26:809-825, In Alfalfa and Alfalfa Improvement, supra), although methods have been developed that help increase productivity and yield (U.S. Pat. No. 4,045,912). Historically, yield and productivity, quality and persistence are objectives of high concern to farmers.

Pasture bloat is a serious and potentially fatal digestive disorder of ruminants which graze on forage legumes. Bloat is a result of the formation of proteinaceous foam that prevents gas escape. Forage diets which contain proanthocyanidins (PA), also called condensed tannins, can help lower the risk of bloating in ruminants by forming a PA-protein complex in the rumen that reduces foam stability and improves the efficiency of dietary protein uptake (Li et al., J. Sci. Food Agric. 70, 89-101 (1996)). Since some non-bloating legumes such as cicer milkvetch (Astragalus cicer L.) do not contain condensed tannins, there are other mechanisms that must also play a role in making some legumes more bloat-safe. At the present time, grazing management is the principal method for controlling bloat incidence in cattle feed or grazed on alfalfa (Berg et al., Canadian Journal of Plant Science, 80, 493-502 (2000); Popp et al., Canadian Journal of Plant Science, 80, 513-517 (2000)).

Tannins had been thought to be anti-nutritional factors which reduce the digestibility of feed. However, more recent studies suggest that tannins actually improve the utilization of feed protein by ruminants without impairing feed intake or carbohydrate digestibility (McMahon et al., Canadian Journal of Plant Science, 80, 469-481 (2000)). Tannins contribute to rumen by-pass of protein thereby enhancing the efficiency of protein utilization in ruminant animals. The tannin-protein complex by-passes or escapes microbial digestion in the rumen, with the protein then becoming available for digestion in the lower intestinal tract where an alkaline pH disrupts the tannin-protein complex. This process is particularly valuable as protein is usually the most expensive component of diets prepared for ruminants (Howarth et al., Antiquality Factors and Normutritive Chemical Compounds 15:493-514, In Alfalfa and Alfalfa Improvement, Hanson et al. (ed.), American Society of Agronomy, Monograph No. 29 (1988).

It has long been held that alfalfa herbage does not contain measurable levels of tannin (see, e.g., Li et al., supra; Gophen et al., Crop Science, 20, 801-804 (1980); R. E. Howarth, Antiquality Factors and Normutritive Chemical Components, 493-514, In Alfalfa and Alfalfa Improvement (1988); Coulman et al., Canadian Journal of Plant Science, 80, 487-491 (2000)). In fact, Coulman et al concluded that it was not possible to produce a leaf-tannin containing alfalfa by conventional selection techniques.

As demonstrated by this review, there is a real and long-felt need for alfalfa varieties containing tannins. The present invention provides alfalfa plants with increased levels of tannins and methods of selection and breeding using such plants. The alfalfa plants provided by this invention will help reduce the risk of bloat in cattle feed or grazed on alfalfa.

### SUMMARY OF THE INVENTION

In one aspect, the present invention can be said to consist of alfalfa germplasm with detectable tannin levels. In another aspect, the present invention can be said to consist of alfalfa germplasm with detectable condensed tannin levels. In yet another aspect, the invention can be said to consist of alfalfa germplasm with detectable tannin levels, wherein the detectable tannins are not the result of inducible expression of tannins.

In another aspect, the present invention can be said to broadly consist in a method for producing bloat-safe alfalfa plants comprising identifying and isolating alfalfa plants which produce tannins.

In still another aspect the invention provides a method for producing bloat-safe alfalfa comprising identifying and isolating alfalfa plants which produce tannins and further crossing these plants with other parental alfalfa plants which do or do not produce tannins so as to produce progeny plants with higher levels of condensed tannins than one or more of the parental plants.

In a further aspect, the invention provides alfalfa plants useful for isolating genes, wherein the expression of the genes results in the production of condensed tannins.

In yet a further aspect, the invention provides plants useful for isolating genes that can be used to produce transgenic plants containing such genes, wherein the expression of the genes results in the transgenic plants producing increased levels of condensed tannins.

The present invention also provides an alfalfa variety having detectable levels of condensed tannin.

In a further aspect, the invention contemplates feed for ruminants comprising alfalfa with detectable levels of tannins. Alfalfa is a basic forage for maximizing ruminant animal production and provides an important source of nutrients for ruminant livestock such as dairy and beef cattle. Feed which includes alfalfa with detectable levels of tannins can take many forms including but not limited to greenchop, silage, hay, haylage, and dehydrated alfalfa, also called dehy. In one embodiment, the invention includes the use of such feed for increasing rumen by-pass of protein within a ruminant.

In another embodiment, the invention also includes using alfalfa with detectable levels of tannin in methods of producing animal feeds and in methods of administering such feeds to animals. In one embodiment, the invention includes using alfalfa with detectable levels of tannin in methods of producing and administering feeds to ruminant animals to increase rumen by-pass of protein.

In one embodiment, the present invention includes seed of alfalfa germplasm designated ‘CW 28061’ which was deposited with the American Type Culture Collection (ATCC) on Oct. 23, 2003 and having ATCC Accession No. 5611.

In one embodiment, the present invention includes seed of alfalfa germplasm designated ‘CW 29053’ which was deposited with the American Type Culture Collection (ATCC) on Oct. 23, 2003 and having ATCC Accession No. 5612.

In another embodiment, the present invention includes seed of a population of alfalfa plants from dormancy groups 2, 3 and/or 4, wherein at least 25% of the plants of the population have a visual staining score greater than 1 when using the DMCA-HCL protocol.

In another embodiment, the present invention includes seed of a population of alfalfa plants from dormancy groups 5, 6, 7, 8 and/or 9, wherein at least 25% of the plants of the population have a visual staining score greater than 1 when using the DMCA-HCL protocol.

In another embodiment, the present invention includes an alfalfa plant, one or more plants cells of an alfalfa plant, one or more plant tissues of an alfalfa plant, or one or more plant parts of an alfalfa plant, wherein the alfalfa plant, plant cell, plant tissue or plant part has a detectable level of condensed tannins. Examples of such plant cells, plant tissues or plant parts include but are not limited to pollen, ovary, ovules, cotyledons, seeds, seedlings, leaflets, leaves, petioles, stems, branches, stipules, and the like.

In another embodiment, the present invention includes an alfalfa plant having all or substantially all of the physiological and morphological characteristics of a population of plants produced by the seed of an alfalfa variety with detectable levels of condensed tannins.

In yet another embodiment, the present invention includes a cell culture or tissue culture of regenerable cells of an alfalfa plant with detectable levels of condensed tannins, wherein the cell culture or tissue culture regenerates plants having all or substantially all of the morphological and physiological characteristics of the alfalfa plant.

In one such embodiment, the cell culture or tissue culture is derived from a plant part selected from the group consisting of leaves, roots, root tips, root hairs, anthers, pistils, stamens, pollen, ovules, flowers, seeds, embryos, stems, buds, cotyledons, hypocotyls, cells and protoplasts.

In another such embodiment, the present invention includes an alfalfa plant regenerated from the above described cell culture or tissue culture.

Another aspect of the present invention is a method for producing first-generation synthetic variety alfalfa seed, the method comprising crossing a first parent alfalfa plant with a second parent alfalfa plant and harvesting the resultant first-generation (F1) hybrid alfalfa seed, wherein said first or second parent alfalfa plant is the alfalfa plant produced by the seed of alfalfa variety having detectable levels of condensed tannins.

In another embodiment, the present invention includes the use of an alfalfa plant with detectable levels of condensed tannins in the breeding and production of alfalfa plants with detectable levels of condensed tannins. The use of such alfalfa plants include using the pollen, ovules, whole plants or regenerable plant parts in the breeding or production of alfalfa plants with detectable levels of condensed tannins.

Although the present invention is broadly as defined above, it will be appreciated by those person skilled in the art that it is not limited thereto and that it further includes the embodiments which are described below.

Further objects and advantages of the present invention will be clear from the description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1I. Photographs of the stained leaves of alfalfa showing the scoring system and the controls. Staining indicates the presence of tannins. See Table 1, below, for a description of the scoring system.

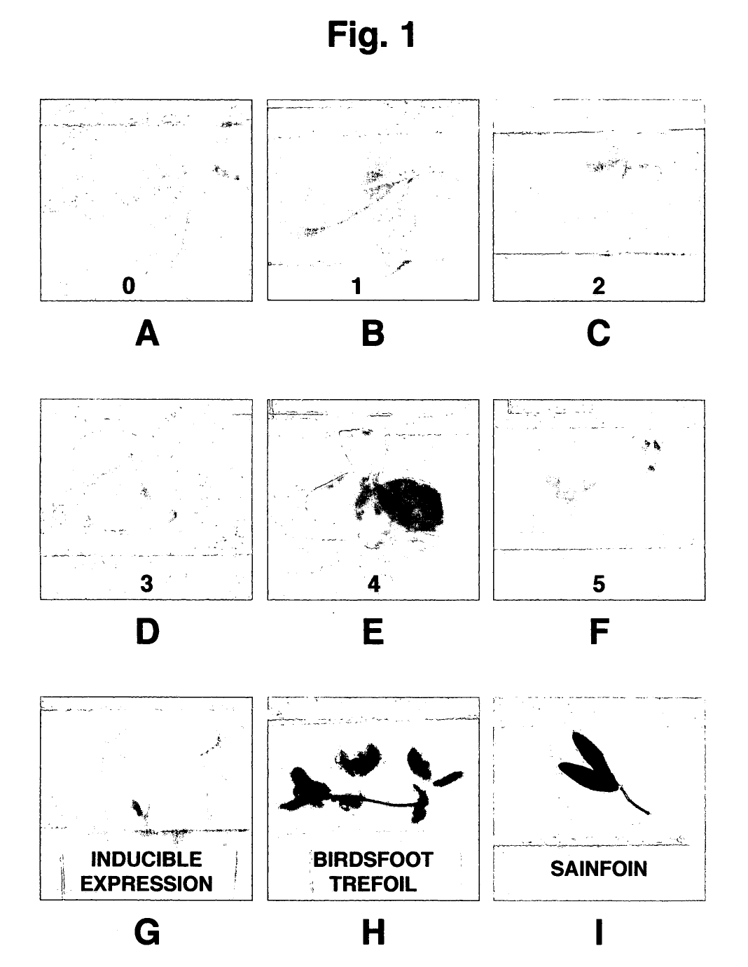

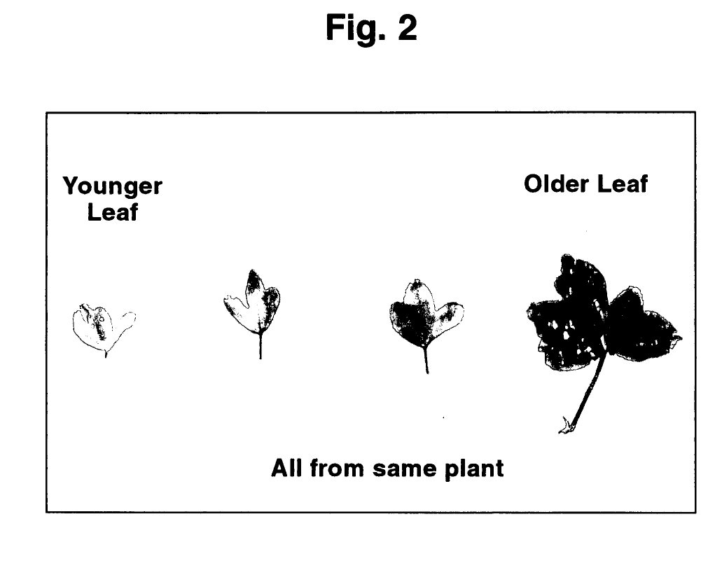

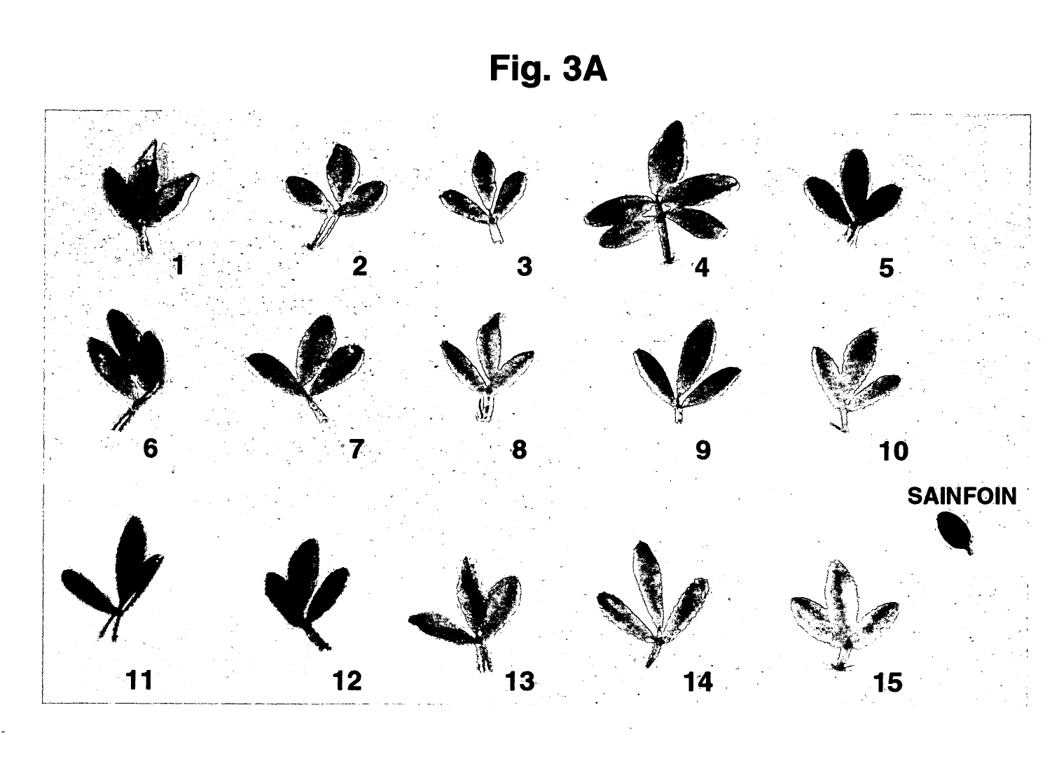

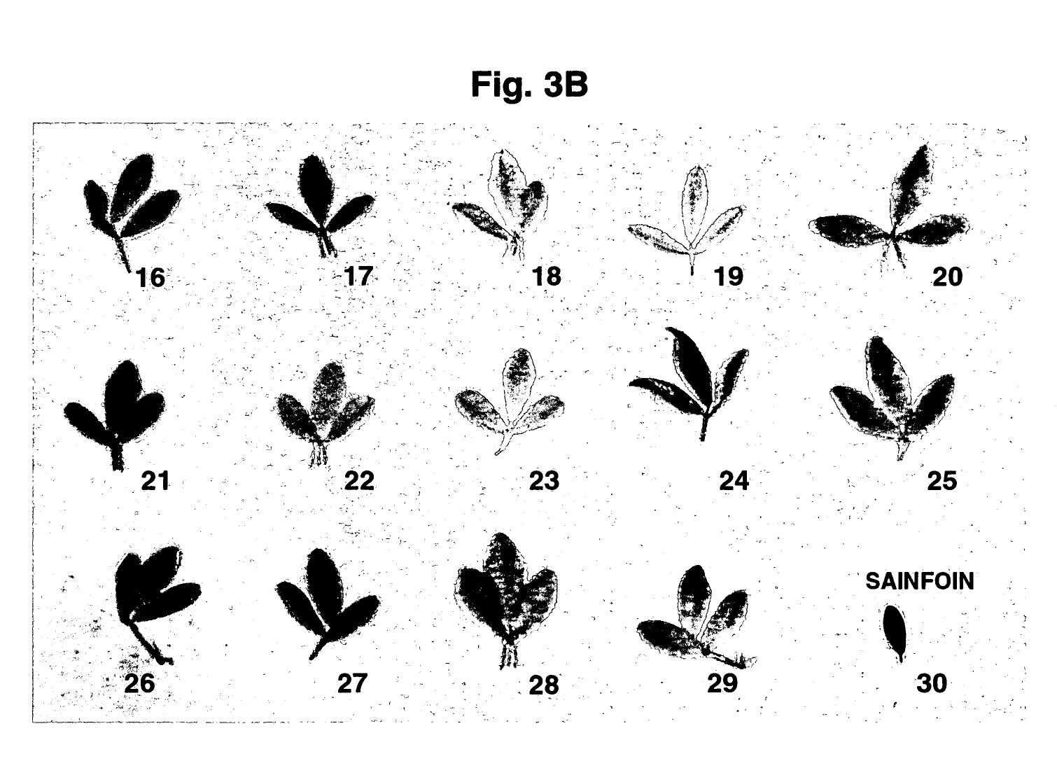

TABLE 1
Explanation of the scoring system for FIG. 1.

| FIG. | Staining Score | Staining |
|---|---|---|
| 1A | 0 (Alfalfa) | None |
| 1B | 1 (Alfalfa) | Very faint blue, spotty |
| 1C | 2 (Alfalfa) | Faint blue, larger spots |
| 1D | 3 (Alfalfa) | Very light blue, covers about ½ to ¾ of |
| | | the non-vein leaf area |
| 1E | 4 (Alfalfa) | Light blue, covers ¾ or more of the leaf area |
| 1F | 5 (Alfalfa) | Moderately light blue, covers entire leaf |
| 1G | Inducible | Localized moderately light blue spots. |
| | (Alfalfa) | |
| 1H | Birdsfoot trefoil | Positive control. Heavy blue, entire leaf and |
| | | stem area |
| 1I | Sainfoin | Positive control. Heavy blue, entire leaf and |
| | | stem area |

FIG. 2. Photograph of the stained leaves of plant Feb. 19, 2002 at different stages of development. All leaves are from the same plant.

FIG. 3A-3B. Photographs of the stained leaves of a random group of 29 alfalfa plants compared to sainfoin. See the Examples section for the scoring of the plants for the presence/absence of tannin.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Overview of the Invention

Pasture bloat in ruminants is caused by the formation of a proteinaceous foam which prevents gas escape from the reticulo-rumen. Tannins prevent bloat by acting as protein precipitants. Tannins also improve the utilization of feed protein by ruminants without impairing feed intake or carbohydrate digestibility and therefore reduce the need for protein supplementation in ruminant diets. The present invention is directed to the development of alfalfa plants with increased tannin levels and methods for identifying and isolating such plants. Furthermore, the invention is directed to the production and use of feeds which include alfalfa plants with increased tannin levels.

Definitions

As used herein, the term “alfalfa” means any Medicago species, including, but not limited to, M. sativa, M. murex, M. falcata, M. prostrata and M. truncatula. Thus, as used herein, the term “alfalfa” means any type of alfalfa including, but is not limited to, any alfalfa commonly referred to as cultivated alfalfa, diploid alfalfa, glanded alfalfa, purple-flowered alfalfa, sickle alfalfa, variegated alfalfa, wild alfalfa, or yellow-flowered alfalfa.

As used herein, the term “tannin” means any one of a group of complex nonuniform plant constituents that can be classified into hydrolyzable tannins (esters of a sugar, usually glucose, and one or several trihydroxybenzene-carboxylic acids) and condensed tannins (derivatives of flavonols). Tannins are used in tanning, dyeing, photography, and as clarifying agents for beer and wine. The term “tannins” is sometimes used synonymously with “tannic acid.” Tannins form black stains in the presence of iron. Stedmans's Medical Dictionary, 27th Edition, pg. 1785 (2001). For a review of tannin chemistry, see, e.g., McMahon et al., Canadian Journal of Plant Science, 80, 469-485 (2000).

As used herein, the term “condensed tannin” means a flavonol-derived water soluble phenolic compound having the property of precipitating proteins. The term “condensed tannin” is sometimes used synonymously with the terms “proanthocyanidin” or “PA.” More specifically, condensed tannins are polymers of flavan-3-ol (e.g., catechin) or flavan-3,4-diol (proanthocyanidins) linked by C—C or C—O—C bonds to yield compounds of varying molecular weight (Leinmuller et al., 9-12, In Animal Research and Development, A. Bittner, ed. Vol. 33, Institut fur Wissenschaftliche Zusammenarbeit, Germany (1991)).

As used herein, the term “transformation” means the transfer of nucleic acid (i.e., a nucleotide polymer) into a cell. As used herein, the term “genetic transformation” means the transfer and incorporation of DNA, especially recombinant DNA, into a cell.

As used herein, the term “transgenic” means cells, cell cultures, plants, and progeny of plants which have received a foreign or modified gene by one of the various methods of transformation, wherein the foreign or modified gene is from the same or different species than the species of the plant receiving the foreign or modified gene.

As used herein, the term “variety” means a subdivision of a species, consisting of a group of individuals within the species which are distinct in form or function from other similar arrays of individuals.

Fall Dormancy (FD). The reaction of alfalfa varieties to decreasing daylength and temperatures in the fall versus check varieties. FD 1=‘Maverick’; FD 2=‘Vernal’; FD 3=‘5246’; FD 4=‘Legend’; FD 5=‘Archer’; FD 6=‘ABI 700’; FD 7=‘Dona Ana’; FD 8=‘Pierce’; FD 9=‘CUF101’; FD 10=‘UC-1887’; and FD 11=‘UC-1465’.

### EXAMPLES

### Example 1

### Initial Screening to Identify Alfalfa Plants with Condensed Tannins

Screening of plants was performed following the DMACA-HCL protocol of Li et al., J. Sci. Food Agric. 70, 89-101 (1996). Initial screenings were conducted using plants from dormancy groups 2 to 4 and approximately 2,000 mature, greenhouse-grown alfalfa plants from dormancy 5 to 9. Approximately 50% of the plants did not stain, approximately 10% stained lightly around areas of leaf damage (indicative of inducible expression), and the rest stained lightly to moderately throughout the whole leaf.

FIGS. 1A-1I and the accompanying Table 1 in the figure description provide the scoring system that was utilized. The intensity of staining was scored on a scale of from “1” to “5”, with “1” being a very faint blue, and with “5” being a moderately light blue (See FIGS. 1B-1F). Some plants were identified which exhibited patterns of staining which are consistent with inducible expression. (See FIG. 1G, Table 1). Unlike the plants scored with a 1, 2, 3, 4 or 5, the observed inducible expressions were associated with tannin expression around a wound site. Birdsfoot trefoil (Lotus corniculatus L.) and sainfoin (Onobrychis viciaefolia Scop.) were included as controls. Birdsfoot trefoil and sainfoin are both known to contain condensed tannins in their foliage and their leaves stain dark blue with this test. See, e.g., Sarkar et al., Crop Science 16, 543-546 (1976); Dalrymple et al., Crop Science 24, 921-923 (1984). On the scale used herein, both species would score at least 10 or 15, as they both stained a very dark blue (see FIGS. 1H and 1I, Table 1).

Approximately 20% of the plants scored “1”; 15% of the plants scored “2”; 10% of the plants scored “3”; 5% of the plants scored “4”; and 1% of the plants scored “5”. The results for a random sample of 29 plants from dormancy groups 2-4 are provided in Table 2.

TABLE 2
Potential Cycle-0 plants from dormancy groups 2-4.
West Salem, WI ES01 Nursery.

| Number | Plant | Score |
|---|---|---|
| 1 | 01.03.01 | 0.5 |
| 2 | 01.04.01 | 0.5 |
| 3 | 01.17.06 | 1.0 |
| 4 | 02.14.09 | 1.0 |
| 5 | 02.19.02 | 3.0 |
| 6 | 02.22.09 | 2.0 |
| 7 | 03.13.08 | 1.5 |
| 8 | 03.19.03 | 0.0 |
| 9 | 03.20.09 | 2.0 |
| 10 | 03.24.01 | 0.0 |
| 11 | 03.29.05 | 0.5 |
| 12 | 03.29.09 | 0.5 |
| 13 | 04.17.02 | 0.0 |
| 14 | 04.29.04 | 0.5 |
| 15 | 05.04.09 | 0.0 |
| 16 | 05.26.01 | 0.5 |
| 17 | 05.27.01 | 2.0 |
| 18 | 05.27.03 | 1.0 |
| 19 | 06.31.10 | 0.0 |
| 20 | 07.15.01 | 1.5 |
| 21 | 07.21.05 | 2.5 |
| 22 | 07.22.09 | 0.0 |
| 23 | 07.28.09 | 0.0 |
| 24 | 08.30.06 | 2.5 |
| 25 | 10.26.07 | 2.0 |
| 26 | 10.26.08 | 0.5 |
| 27 | 09.21.07 | 2.0 |
| 28 | 09.31.05 | 1.5 |
| 29 | 10.07.04 | 1.5 |
| 30 | Sainfoin | 10.0 |

It was noted during the staining protocol that staining would frequently occur only on around damage on a leaf. This pattern of staining is consistent with inducible expression of involved in the synthesis of tannins.

It was also noted that plants tended to stain more darkly and the percentage of plants that stained increased as the temperature at which the plants were grown increased. This indicates that all of the plants in any one test should be grown at the same temperature.

In an effort to account for further possible sources of experimental error due to environmental and plant variation, additional tests were conducted to determine whether the stage of growth of the leaf affected the results that were obtained. It was noted that very young leaves stained more lightly, while older leaves did not clear well making the stain difficult to read (FIG. 2). However, all moderately aged leaves were similar in staining intensity. The temperature of the leaf at the time of harvest did not appear to effect the staining results obtained using the moderately aged leaves, even when harvested at cooler temperatures.

With the above parameters in mind, plants that stained most lightly or most darkly were re-tested to ensure that temperature and leaf stage were not factors that influenced the results that had been obtained.

Alfalfa seedlings were also screened. It was very difficult to find any staining with very young seedlings, but 4 to 6 week old seedlings were found to be suitable for testing.

### Example 2

### Breeding for Alfalfa Plants with Condensed Tannins: Cycle 0

From the initial screening described above, several hundred plants were found that scored “3” or higher. The highest scoring lines were chosen to constitute the Cycle-0 plants, with selection intensity being approximately 15%. In each crossing group approximately 50-60 plants were polycrossed, except for dormancy/crossing group 5 for which there were insufficient plants to make 50 crosses. Table 3 summarizes selected crosses of Cycle-0 plants that resulted in Cycle-1 seed, wherein the assigned staining levels are from the second screen when temperatures were warm.

TABLE 3
Selected parent plants for Cycle-0 and crosses resulting in Cycle-1 seed.
Dormancy Groups 5-9.

| | | | |
|---|---|---|---|
| | | Number of | % |
| Parent Plants | Nursery Source | plants | contribution |

Dormancy 9

| | | | |
|---|---|---|---|
| 89139 | SYN | 1 | 1.6 |
| 99111 | SYN | 2 | 3.1 |
| 00052 | cages | 1 | 1.6 |
| 89132 | FDN | 1 | 1.6 |
| 59125 | MFS | 2 | 3.1 |
| 89132 | VFS | 1 | 1.6 |
| 6975 | WFS | 1 | 1.6 |
| 79056 | SYN | 1 | 1.6 |
| 79063 | SYN | 1 | 1.6 |
| 79083 | SYN | 1 | 1.6 |
| 79103 | SYN | 1 | 1.6 |
| 89139 | SYN | 2 | 3.1 |
| 99060 | SYN | 1 | 1.6 |
| 99111 | SYN | 1 | 1.6 |
| 00083 | cages | 1 | 1.6 |
| 00086 | cages | 1 | 1.6 |
| 00092 | cages | 1 | 1.6 |
| 4958 | MFS | 1 | 1.6 |
| 69120 | MFS | 1 | 1.6 |
| 89061 | SN | 1 | 1.6 |
| 99113 | SN | 1 | 1.6 |
| 69117 | VFS | 1 | 1.6 |
| 79103 | VFS | 1 | 1.6 |
| 79116 | VFS | 1 | 1.6 |
| 6956 | WFS | 1 | 1.6 |
| 59125 | WFS | 1 | 1.6 |
| 79056 | SYN | 1 | 1.6 |
| 79083 | SYN | 2 | 3.1 |
| 89064 | SYN | 1 | 1.6 |
| 89139 | SYN | 1 | 1.6 |
| 99057 | SYN | 2 | 3.1 |
| 99060 | SYN | 1 | 1.6 |
| 99111 | SYN | 6 | 9.4 |
| 99113 | SYN | 1 | 1.6 |
| 99114 | SYN | 1 | 1.6 |
| 99116 | SYN | 3 | 4.7 |
| 00051 | cages | 1 | 1.6 |
| 00086 | cages | 1 | 1.6 |
| 00092 | cages | 1 | 1.6 |
| 49100 SYN halfsibs | SPN | 1 | 1.6 |
| 4958 SYN halfsibs | SPN | 1 | 1.6 |
| 89132 | FDN | 1 | 1.6 |
| 89132 | FDN | 1 | 1.6 |
| 89066 | HFS | 1 | 1.6 |
| 59128 | MFS | 1 | 1.6 |
| 79123 | NRKN | 1 | 1.6 |
| 79123 | NRKN | 1 | 1.6 |
| 79117 | VFS | 1 | 1.6 |
| 79123 | VFS | 1 | 1.6 |
| 89132 | VFS | 1 | 1.6 |
| 6975 | WFS | 1 | 1.6 |
| 59125 | WFS | 1 | 1.6 |
| Crossing Group Total | | 64 | 100.0 |
| 89139 | SYN | 1 | 11.1 |
| 99111 | SYN | 2 | 22.2 |
| 00052 | cages | 1 | 11.1 |
| 89132 | FDN | 1 | 11.1 |
| 59125 | MFS | 2 | 22.2 |
| 89132 | VFS | 1 | 11.1 |
| 6975 | WFS | 1 | 11.1 |
| Crossing Group Total | | 9 | 100.0 |
| 4958 | MFS | 1 | 3.7 |
| 6956 | WFS | 1 | 3.7 |
| 59125 | WFS | 1 | 3.7 |
| 69112 | WFS | 1 | 3.7 |
| 69117 | VFS | 1 | 3.7 |
| 69120 | MFS | 1 | 3.7 |
| 79056 | SYN | 1 | 3.7 |
| 79063 | SYN | 1 | 3.7 |
| 79083 | SYN | 1 | 3.7 |
| 79084 | VFS | 1 | 3.7 |
| 79103 | SYN | 1 | 3.7 |
| 79103 | VFS | 1 | 3.7 |
| 79116 | VFS | 1 | 3.7 |
| 89061 | SN | 1 | 3.7 |
| 89139 | SYN | 2 | 7.4 |
| 99060 | SYN | 1 | 3.7 |
| 99111 | SYN | 2 | 7.4 |
| 99113 | SN | 1 | 3.7 |
| 99113 | SYN | 1 | 3.7 |
| 00083 | cages | 2 | 7.4 |
| 00086 | cages | 1 | 3.7 |
| 00092 | cages | 2 | 7.4 |
| 97-404 | SPN | 1 | 3.7 |
| Crossing Group Total | | 27 | 100.0 |
| 6972 | WFS | 1 | 1.9 |
| 6975 | WFS | 1 | 1.9 |
| 59125 | WFS | 1 | 1.9 |
| 59128 | MFS | 1 | 1.9 |
| 69112 | HFS | 1 | 1.9 |
| 69117 | MFS | 1 | 1.9 |
| 69118 | WFS | 1 | 1.9 |
| 69120 | NRKN | 1 | 1.9 |
| 79056 | SYN | 2 | 3.8 |
| 79083 | SYN | 2 | 3.8 |
| 79085 | VFS | 1 | 1.9 |
| 79087 | SYN | 1 | 1.9 |
| 79103 | SYN | 1 | 1.9 |
| 79117 | VFS | 1 | 1.9 |
| 79123 | NRKN | 2 | 3.8 |
| 79123 | VFS | 3 | 5.8 |
| 89061 | SN | 1 | 1.9 |
| 89064 | SYN | 2 | 3.8 |
| 89066 | HFS | 1 | 1.9 |
| 89132 | FDN | 2 | 3.8 |
| 89132 | VFS | 1 | 1.9 |
| 89139 | SYN | 1 | 1.9 |
| 99057 | SYN | 2 | 3.8 |
| 99060 | SYN | 1 | 1.9 |
| 99111 | SYN | 8 | 15.4 |
| 99113 | SYN | 2 | 3.8 |
| 99114 | SYN | 1 | 1.9 |
| 99116 | SYN | 3 | 5.8 |
| 00051 | cages | 1 | 1.9 |
| 00052 | cages | 1 | 1.9 |
| 00086 | cages | 1 | 1.9 |
| 00092 | cages | 1 | 1.9 |
| 49100 SYN halfsibs | SPN | 1 | 1.9 |
| 4958 SYN halfsibs | SPN | 1 | 1.9 |
| Crossing Group Total | | 52 | 100.0 |
| 5965 | IVFS | 1 | 25.0 |
| 79064 | HFS | 1 | 25.0 |
| DK-191 | IVFS | 2 | 50.0 |
| Crossing Group Total | | 4 | 100.0 |

Dormancy 8

| | | | |
|---|---|---|---|
| 3863 | MoFS | 1 | 2.6 |
| 3864 | MoFS | 1 | 2.6 |
| 3869 | MoFS | 1 | 2.6 |
| 4879 | WFS | 1 | 2.6 |
| 4880 | WFS | 1 | 2.6 |
| 4880 | KACFS | 1 | 2.6 |
| 6881 | VFS | 1 | 2.6 |
| 58127 | MFS | 1 | 2.6 |
| 68115 | MFS | 1 | 2.6 |
| 77095 | 8-SPN | 1 | 2.6 |
| 78059 | VFS | 1 | 2.6 |
| 78066 | 8-SPN | 1 | 2.6 |
| 78088 | SYN | 1 | 2.6 |
| 78089 | SPN | 1 | 2.6 |
| 78101 | SPN | 1 | 2.6 |
| 78122 | FDN | 1 | 2.6 |
| 88077 | HFS | 1 | 2.6 |
| 88122 | SYN | 1 | 2.6 |
| 88123 | SYN | 1 | 2.6 |
| 98080 | SYN | 1 | 2.6 |
| 98090 | SYN | 1 | 2.6 |
| 98110 | SYN | 1 | 2.6 |
| 98118 | SYN | 1 | 2.6 |
| 98119 | SYN | 1 | 2.6 |
| 00059 | cages | 2 | 5.1 |
| 00080 | cages | 1 | 2.6 |
| 00093 | cages | 1 | 2.6 |
| 4890 SYN half sibs | SPN | 2 | 5.1 |
| 69120 SYN half sibs | SPN | 2 | 5.1 |
| 88SWR | KACFS | 1 | 2.6 |
| 88SWR | WFS | 1 | 2.6 |
| 97-473 | SPN | 1 | 2.6 |
| 99-497 | SN | 3 | 7.7 |
| unknown | SPN | 1 | 2.6 |
| Crossing Group Total | | 39 | 100.0 |
| 88123 | SYN | 1 | 14.3 |
| 77095 | SPN | 1 | 14.3 |
| 4890 SYN half sibs | SPN | 1 | 14.3 |
| unknown | SPN | 1 | 14.3 |
| 00093 | cages | 1 | 14.3 |
| 88077 | HFS | 1 | 14.3 |
| unknown | unknown | 1 | 14.3 |
| Crossing Group Total | | 7 | 100.0 |
| 3863 | MoFS | 1 | 4.0 |
| 4880 | WFS | 1 | 4.0 |
| 6868 | VFS | 1 | 4.0 |
| 6881 | VFS | 1 | 4.0 |
| 58127 | MFS | 1 | 4.0 |
| 68115 | MoFS | 1 | 4.0 |
| 78059 | VFS | 1 | 4.0 |
| 78066 | SPN | 1 | 4.0 |
| 78088 | SYN | 1 | 4.0 |
| 78101 | SYN | 1 | 4.0 |
| 78122 | FDN | 1 | 4.0 |
| 98080 | SYN | 1 | 4.0 |
| 98110 | SYN | 2 | 8.0 |
| 98117 | SYN | 1 | 4.0 |
| 98118 | SYN | 1 | 4.0 |
| 00058 | cages | 2 | 8.0 |
| 69120 SYN half sibs | SPN | 1 | 4.0 |
| 88SWR | WFS | 1 | 4.0 |
| 97-473 | SPN | 1 | 4.0 |
| 99-497 | SN | 3 | 12.0 |
| Prestige | WFS | 1 | 4.0 |
| Crossing Group Total | | 25 | 100.0 |
| 3863 | MoFS | 1 | 2.8 |
| 3864 | MoFS | 1 | 2.8 |
| 3869 | MoFS | 1 | 2.8 |
| 4879 | WFS | 1 | 2.8 |
| 4880 | KACFS | 1 | 2.8 |
| 4880 | MoFS | 1 | 2.8 |
| 4880 | WFS | 1 | 2.8 |
| 5875 | HFS | 1 | 2.8 |
| 68115 | MFS | 1 | 2.8 |
| 68115 | NRKN | 1 | 2.8 |
| 68116 | WFS | 1 | 2.8 |
| 78089 | SPN | 1 | 2.8 |
| 78101 | SPN | 1 | 2.8 |
| 78101 | VFS | 1 | 2.8 |
| 78122 | HFS | 1 | 2.8 |
| 88122 | SYN | 1 | 2.8 |
| 98090 | SYN | 1 | 2.8 |
| 98110 | SYN | 1 | 2.8 |
| 98119 | SYN | 1 | 2.8 |
| 00055 | cages | 1 | 2.8 |
| 00056 | cages | 1 | 2.8 |
| 00059 | cages | 2 | 5.6 |
| 00080 | cages | 2 | 5.6 |
| 3864 SYN halfsibs | SPN | 1 | 2.8 |
| 4880 SYN halfsibs | SPN | 1 | 2.8 |
| 4890 SYN halfsibs | SPN | 1 | 2.8 |
| 4978 SYN halfsibs | SPN | 1 | 2.8 |
| 69120 SYN half sibs | SPN | 1 | 2.8 |
| 88SWR | KACFS | 1 | 2.8 |
| 88SWR | WFS | 1 | 2.8 |
| 97-439 | SPN | 1 | 2.8 |
| 99-497 | SN | 1 | 2.8 |
| 99-497 | SN | 2 | 5.6 |
| Crossing Group Total | | 36 | 100.0 |
| 69117 | GRZ | 1 | 7.1 |
| 79063 | GRZ | 1 | 7.1 |
| 79064 | GRZ | 1 | 7.1 |
| 79085 | GRZ | 1 | 7.1 |
| 79086 | GRZ | 1 | 7.1 |
| 79103 | GRZ | 1 | 7.1 |
| 79115 | GRZ | 1 | 7.1 |
| 79123 | GRZ | 1 | 7.1 |
| 89063 | GRZ | 1 | 7.1 |
| 89068 | GRZ | 1 | 7.1 |
| 89071 | GRZ | 1 | 7.1 |
| 89139 | GRZ | 1 | 7.1 |
| 89140 | GRZ | 1 | 7.1 |
| unknown | GRZ | 1 | 7.1 |
| Crossing Group Total | | 14 | 100.0 |
| 12-34 | salt | 1 | 14.3 |
| 5875 | salt | 1 | 14.3 |
| 88055 | HFS | 1 | 14.3 |
| 88135 | salt | 1 | 14.3 |
| 88136 | salt | 1 | 14.3 |
| 98102 | HFS | 1 | 14.3 |
| 99-497 | NRKN | 1 | 14.3 |
| Crossing Group Total | | 7 | 100.0 |
| 6954 | GRZ | 1 | 7.7 |
| 68115 | Salt | 1 | 7.7 |
| 88097 | HFS | 1 | 7.7 |
| 88074 | HFS | 1 | 7.7 |
| 88123 | salt | 1 | 7.7 |
| 88135 | HFS | 1 | 7.7 |
| 98117 | HFS | 1 | 7.7 |
| 98117 | salt | 2 | 15.4 |
| 98118 | HFS | 1 | 7.7 |
| 99-497 | NRKN | 3 | 23.1 |
| Crossing Group Total | | 13 | 100.0 |

Dormancy 7

| | | | |
|---|---|---|---|
| 2870 | WFS | 1 | 3.4 |
| 3765 | MoFS | 1 | 3.4 |
| 5666 | MFS | 1 | 3.4 |
| 6683 | MFS | 1 | 3.4 |
| 6779 | MFS | 1 | 3.4 |
| 6780 | MFS | 1 | 3.4 |
| 6789 | FDN | 1 | 3.4 |
| 6790 | MFS | 1 | 3.4 |
| 57104 | NRKN | 1 | 3.4 |
| 57115 | VW | 1 | 3.4 |
| 77093 | VFS | 1 | 3.4 |
| 77121 | VFS | 1 | 3.4 |
| 87103 | SN | 2 | 6.9 |
| 87105 | HFS | 1 | 3.4 |
| 87106 | HFS | 1 | 3.4 |
| 87129 | VFS | 2 | 6.9 |
| 00064 | cages | 2 | 6.9 |
| 4.152.4 | SPN | 1 | 3.4 |
| 4.170.7 | SPN | 1 | 3.4 |
| 5.135.9 | SPN | 1 | 3.4 |
| 5.69.8 | SPN | 1 | 3.4 |
| 5.76.8 | SPN | 1 | 3.4 |
| 7.13.4 | SYN | 1 | 3.4 |
| 7.19.6 | SYN | 1 | 3.4 |
| 7.20.6 | SYN | 1 | 3.4 |
| Achiever | MoFS | 1 | 3.4 |
| Crossing Group Total | | 29 | 100.0 |
| 4887 | HFS | 1 | 20.0 |
| 87103 | SN | 1 | 20.0 |
| 87129 | VFS | 1 | 20.0 |
| 4.152.4 | SPN | 1 | 20.0 |
| 7.13.4 | SYN | 1 | 20.0 |
| Crossing Group Total | | 5 | 100.0 |
| 2870 | WFS | 1 | 6.7 |
| 6780 | MFS | 1 | 6.7 |
| 6789 | FDN | 1 | 6.7 |
| 6790 | MFS | 1 | 6.7 |
| 57104 | NRKN | 1 | 6.7 |
| 57115 | VW | 1 | 6.7 |
| 77093 | VFS | 1 | 6.7 |
| 77121 | VFS | 1 | 6.7 |
| 87103 | SN | 1 | 6.7 |
| 87106 | HFS | 1 | 6.7 |
| 87129 | VFS | 1 | 6.7 |
| 00064 | cages | 1 | 6.7 |
| 4.152.8 | SPN | 1 | 6.7 |
| 5.135.9 | SPN | 1 | 6.7 |
| 5.76.8 | SPN | 1 | 6.7 |
| Crossing Group Total | | 15 | 100.0 |
| 3765 | MoFS | 1 | 5.9 |
| 5666 | MFS | 1 | 5.9 |
| 6683 | MFS | 1 | 5.9 |
| 6779 | MFS | 1 | 5.9 |
| 57102 | HFS | 1 | 5.9 |
| 57104 | BW | 1 | 5.9 |
| 57104 | HFS | 1 | 5.9 |
| 77068 | VFS | 1 | 5.9 |
| 87105 | HFS | 1 | 5.9 |
| 87129 | VFS | 1 | 5.9 |
| 00064 | cages | 1 | 5.9 |
| 4.170.7 | SPN | 1 | 5.9 |
| 5.69.8 | SPN | 1 | 5.9 |
| 7.19.6 | SYN | 1 | 5.9 |
| 7.20.6 | SYN | 1 | 5.9 |
| Achiever | MoFS | 1 | 5.9 |
| Sutter | WFS | 1 | 5.9 |
| Crossing Group Total | | 17 | 100.0 |
| 77068 | HFS | 1 | 33.3 |
| 87129 | HFS | 1 | 33.3 |
| 96122 | HFS | 1 | 33.3 |
| Crossing Group Total | | 3 | 100.0 |

Dormancy 6

| | | | |
|---|---|---|---|
| 3567 | MoFS | 1 | 2.9 |
| 3568 | MoFS | 2 | 5.9 |
| 5567 | HFS | 1 | 2.9 |
| 5782 | NRKN | 1 | 2.9 |
| 5783 | NRKN | 2 | 5.9 |
| 5783 | unknown | 1 | 2.9 |
| 5799 | unknown | 1 | 2.9 |
| 5885 | WFS | 1 | 2.9 |
| 6699 | FDN | 1 | 2.9 |
| 6699 | SN | 1 | 2.9 |
| 6699 | NRKN | 2 | 5.9 |
| 76098 | SN | 1 | 2.9 |
| 76120 | VFS | 1 | 2.9 |
| 86120 | HFS | 2 | 5.9 |
| 86128 | VFS | 2 | 5.9 |
| 86128 | NRKN | 1 | 2.9 |
| 96122 | SN | 3 | 8.8 |
| 00069 | cages | 1 | 2.9 |
| 4.14.6 | SPN | 1 | 2.9 |
| 5.122.2 | SPN | 1 | 2.9 |
| 5.153.10 | SPN | 1 | 2.9 |
| 5.153.8 | SPN | 1 | 2.9 |
| 7.5.9 | SYN | 1 | 2.9 |
| 7.6.10 | SYN | 1 | 2.9 |
| 7.6.2 | SYN | 1 | 2.9 |
| 7.6.4 | SYN | 1 | 2.9 |
| 7.7.11 | SYN | 1 | 2.9 |
| | | 34 | 100.0 |
| 3567 | MoFS | 1 | 16.7 |
| 5782 | NRKN | 1 | 16.7 |
| 5783 | NRKN | 1 | 16.7 |
| 6699 | FDN | 1 | 16.7 |
| 6699 | SN | 1 | 16.7 |
| 96122 | SN | 1 | 16.7 |
| Crossing Group Total | | 6 | 100.0 |
| 3567 | MoFS | 1 | 5.0 |
| 3568 | MoFS | 2 | 10.0 |
| 3673 | MoFS | 1 | 5.0 |
| 5567 | HFS | 1 | 5.0 |
| 5799 | unknown | 1 | 5.0 |
| 6699 | NRKN | 1 | 5.0 |
| 6699 | FDN | 1 | 5.0 |
| 56108 | WFS | 1 | 5.0 |
| 66101 | WFS | 1 | 5.0 |
| 76106 | HFS | 1 | 5.0 |
| 86079 | HFS | 1 | 5.0 |
| 86120 | HFS | 1 | 5.0 |
| 96122 | SN | 2 | 10.0 |
| 5.153.10 | SPN | 1 | 5.0 |
| 5.153.8 | SPN | 1 | 5.0 |
| 7.10.13 | SYN | 1 | 5.0 |
| 7.6.10 | SYN | 1 | 5.0 |
| 7.6.2 | SYN | 1 | 5.0 |
| Crossing Group Total | | 20 | 100.0 |
| 5567 | WFS | 1 | 3.7 |
| 5567 | NRKN | 1 | 3.7 |
| 5783 | NRKN | 2 | 7.4 |
| 5885 | WFS | 1 | 3.7 |
| 6699 | NRKN | 1 | 3.7 |
| 6699 | WFS | 1 | 3.7 |
| 66110 | WFS | 1 | 3.7 |
| 76098 | SN | 1 | 3.7 |
| 76120 | VFS | 2 | 7.4 |
| 86110 | HFS | 1 | 3.7 |
| 86120 | HFS | 1 | 3.7 |
| 86128 | VFS | 2 | 7.4 |
| 86128 | NRKN | 1 | 3.7 |
| 86128 | FDN | 1 | 3.7 |
| 96122 | SN | 2 | 7.4 |
| 00069 | cages | 2 | 7.4 |
| 00075 | cages | 1 | 3.7 |
| 4.14.6 | SPN | 1 | 3.7 |
| 5.122.2 | SPN | 1 | 3.7 |
| 7.5.9 | SYN | 1 | 3.7 |
| 7.6.4 | SYN | 1 | 3.7 |
| 7.7.11 | SYN | 1 | 3.7 |
| Crossing Group Total | | 27 | 100.0 |
| 75097 | HFS | 1 | 14.3 |
| 76120 | HFS | 1 | 14.3 |
| 86078 | HFS | 2 | 28.6 |
| 86112 | HFS | 1 | 14.3 |
| 86120 | HFS | 1 | 14.3 |
| 95094 | HFS | 1 | 14.3 |
| Crossing Group Total | | 7 | 100.0 |
| 6699 | HFS | 1 | 14.3 |
| 65064 | HFS | 1 | 14.3 |
| 85083 | HFS | 1 | 14.3 |
| 85119 | HFS | 2 | 28.6 |
| 86112 | HFS | 1 | 14.3 |
| 95094 | HFS | 1 | 14.3 |
| Crossing Group Total | | 7 | 100.0 |

Dormancy 5

| | | | |
|---|---|---|---|
| 6539 | SN | 1 | 25.0 |
| 5681 | FDN | 1 | 25.0 |
| 6588 | WFS | 1 | 25.0 |
| 6539 | WFS | 1 | 25.0 |
| Crossing Group Total | | 4 | 100.0 |
| 5678 | WFS | 1 | 50.0 |
| 75077 | MFS | 1 | 50.0 |
| Crossing Group Total | | 2 | 100.0 |

Parent Plant = The source of the parent plant. For instance the first plant listed was selected out of breeders seed population CW 89139.
Nursery Source = The nursery that plants were selected from.
% contribution = The percentage of this crossing group that came from this plant or group of plants with the same description.
Note:
Plants were not exclusive to each group.

### Example 3

### Breeding for Alfalfa Plants with Condensed Tannins: Cycle 1

The seed produced from the Cycle-0 crosses (i.e., Cycle-1 seed) was harvested and several hundred seeds were subsequently planted for each of the dormancy/crossing groups. The resultant plants (Cycle-1 plants) were screened at the 4 to 6 week old seedlings stage, or after the first cutting. Thirty Cycle-0 plants were included as comparison checks. The percentage of plants that scored 3 or higher had significantly increased, from about 15% to about 25%.

### Example 4

### Breeding for Alfalfa Plants with Condensed Tannins: Cycle 2

Cycle-1 plants were chosen that had a staining score of 3, 4 or 5. A selection intensity of 25% was used for each group. If a tannin screening run had more than 25% of the plants in the 3, 4 or 5 group, then all plants with a borderline 2/3 score were rechecked to maintain the selection intensity at 25%. The number of selected plants utilized in the Cycle-2 summer greenhouse crosses was 117, while the number of selected plants utilized in the Cycle-2 crosses that went to breeder seed was 182 in one crossing cage (‘CW 29053’) and 212 in the second crossing cage (‘CW 28061’).

Field Characterization Tests for Tannin Expression. Plants were sampled from six entries in an alfalfa yield trial. The six entries included the two experimental alfalfa varieties with detectable tannin levels (i.e., ‘CW 28061’ and ‘CW 29053’) and four check varieties (i.e., ‘Weston’, ‘CW 50073’, ‘CW 3958’ and ‘CW 89064’). Since there was not an exact CYCLE-0 population to use as a check, commercial and standard entries were chosen to constitute the CYCLE-0 populations for each dormancy group.

The trial was planted on April 7 and the first cut was made on June 17. Twenty five stems were randomly sampled from each of four replications one month after the first cut, providing a total of 100 stem samples The first fully expanded leaf from each sampled stem was tested for tannin expression. Generally, this resulted in sampling a leaf that was 2 to 3 leaves down from the apical growing point. Growing conditions were typical for California: hot and dry, with typical daytime temperatures reaching 90 to 100 degrees F. and nighttime temperatures between 60 and 70 degrees F. The plots were irrigated twice per cut using sprinkler irrigation, using about three inches of water per irrigation. The results of the tannin expression assays is provided in Table 4.

TABLE 4
Tannin Field Characterization Test Results.

| | | Percentage of Stems with Stained Leaves | | | | | | | Total | |
|---|---|---|---|---|---|---|---|---|---|---|
| | | Grouped by Staining Score | | | | | | | % for | Average |
| Variety | Description | 0 | 1 | 2 | 3 | 4 | 5 | 6 | 3, 4, 5, 6 | Score |
| CW 28061 | Dormancy 8 | 20 | 31 | 23 | 12 | 10 | 3 | 1 | 26 | 1.74 |
| | Cycle-2 | | | | | | | | | |
| | population | | | | | | | | | |
| Weston | Typical | 44 | 27 | 14 | 10 | 4 | 1 | 0 | 15 | 1.06 |
| | dormancy 8 | | | | | | | | | |
| | Cycle-0 | | | | | | | | | |
| CW 50073 | Typical | 36 | 29 | 19 | 11 | 5 | 0 | 0 | 16 | 1.20 |
| | dormancy 8 | | | | | | | | | |
| | Cycle-0 | | | | | | | | | |
| AVERAGE OF 2 | | 40 | 28 | 16.5 | 10.5 | 4.5 | 0.5 | 0 | 15.5 | 1.13 |
| Dormancy 8 CYCLE-0 | | | | | | | | | | |
| entries | | | | | | | | | | |
| CW 29053 | Dormancy 9 | 20 | 27 | 25 | 16 | 4 | 8 | 0 | 28 | 1.81 |
| | Cycle-2 | | | | | | | | | |
| | population | | | | | | | | | |
| CW 3958 | Typical | 23 | 32 | 27 | 8 | 5 | 5 | 0 | 18 | 1.55 |
| | dormancy 9 | | | | | | | | | |
| | Cycle-0 | | | | | | | | | |
| CW 89064 | Typical | 45 | 35 | 13 | 6 | 0 | 0 | 1 | 7 | 0.85 |
| | dormancy 9 | | | | | | | | | |
| | Cycle-0 | | | | | | | | | |

| | | | | | | | | | |
|---|---|---|---|---|---|---|---|---|---|
| AVERAGE OF 2 | 34 | 33.5 | 20 | 7 | 2.5 | 2.5 | 0.5 | 12.5 | 1.2 |
| Dormancy 9 Cycle-0 | | | | | | | | | |
| entries | | | | | | | | | |
| =Grnd MN | 31.3 | 30.2 | 20.2 | 10.5 | 4.7 | 2.8 | 0.3 | 18.3 | 1.4 |
| =LSD | 12.0 | 11.3 | 8.7 | 9.7 | 6.2 | 4.9 | 1.5 | 10.5 | 0.3 |
| =CV | 25.4 | 24.9 | 28.6 | 61.1 | 88.1 | 114.4 | 302.4 | 37.9 | 16.7 |
| =F trt | 8.8 | 0.7 | 4.1 | 1.2 | 2.4 | 3.9 | 1.1 | 4.9 | 11.6 |
| =F rep | 2.2 | 0.6 | 0.2 | 0.1 | 0.1 | 0.7 | 0.2 | 0.4 | 1.3 |
| R2 | 78.4 | 36.2 | 53.1 | 26.7 | 40.6 | 58.2 | 27.3 | 58.8 | 78.6 |

Greenhouse Characterization Tests for Tannin Expression. Seeds were planted into inserts each with 72 cones (6×12) that fit into standard 10-20 flats. Each cone is about 4 cm in diameter and about 6 cm deep. On December 1, four rows (24 cones) were planted per entry and subsequently thinned to 1 plant per cone. The plants were clipped twice before the first sampling run. The soil was a typical soil-less media (peat moss, perlite, etc.) and standard fertilization was followed (i.e., once every two weeks using a 15-30-15 (with micronutrients) soluble fertilizer). Light was supplemented to reach a total of 16 hours of daylight (i.e., 16 hour day length).

For the first data collection, plants were sampled following about 4 weeks of growth after the second clip-off on February 20. Plants were scored from March 18-21, with a complete replication being scored each day. Typical greenhouse temperatures during the 4 weeks of re-growth were about 60 to 65 at night and about 75 to 85 on sunny days (90% of days) and 65 to 70 on cloudy and rainy days (about 10%).

For the second data collection, plants were sampled about 20 days of growth after the third clipoff on March 22. Plants were scored from April 10-11. Typical greenhouse temperatures during the 3 weeks of re-growth were about 65 at night and about 85 on sunny days (95% of days) and 70 on cloudy and rainy days (about 5%).

Two Cycle-0 populations were utilized for each dormancy group because the selected populations came not from a single variety but rather from a combination of germplasm that is best represented by the average of the two Cycle-0 populations from each dormancy group. Thus, different Cycle-0 entries were used in the greenhouse and the field tests: 1) as described above, the check entries for the yield trials were those that best represented the Cycle-0 germplasm; and 2) the check entries used in the greenhouse test were considered to be the most representative populations.

Table 5 provides summary averages data from the 2 different sets of data from the same plants.

TABLE 5
Tannin Greenhouse Characterization Test Results

| | | | | | | | | | Data | | |
|---|---|---|---|---|---|---|---|---|---|---|---|
| | | | | | | | | | Combined | | |
| | | | | | | | | | by Range of | | |
| | | | | | | | | | Staining | | |
| | | Percentage of Plants with Stained Leaves | | | | | | | Score | | |
| | | Grouped by Staining Score | | | | | | | | % = 3 | Average |
| Variety | | % 0-0.5's | % 6-1.5's | % 1.6-2.5's | % 2.6-3.5's | % 3.6-4.5's | % 4.6-5.5's | %>5.6 | % <3 | or >3 | Score |
| CW | FD 9 | 14.4 | 21.2 | 24.2 | 13.3 | 26.9 | 0.0 | 0.0 | 59.8 | 40.2 | 2.43 |
| 29053 | Tannin | | | | | | | | | | |
| | Cycle-2 | | | | | | | | | | |
| CW | FD 9 | | | | | | | | | | |
| 09052 | Tannin | 25.2 | 25.4 | 22.6 | 15.1 | 10.3 | 1.5 | 0.0 | 73.2 | 26.8 | 1.84 |
| | Cycle-0 | | | | | | | | | | |
| CW | FD 9 | 48.6 | 12.6 | 18.3 | 16.8 | 1.8 | 1.9 | 0.0 | 79.5 | 20.5 | 1.35 |
| 89064 | Tannin | | | | | | | | | | |
| | Cycle-0 | | | | | | | | | | |

| AVERAGE OF 2 | | 36.9 | 19.0 | 20.4 | 16.0 | 6.0 | 1.7 | 0.0 | 76.3 | 23.7 | 1.59 |
|---|---|---|---|---|---|---|---|---|---|---|---|
| Dormancy 9 | | | | | | | | | | | |
| Cycle-0 entries | | | | | | | | | | | |
| CW | FD 8 | 15.5 | 23.4 | 23.1 | 13.3 | 24.7 | 0.0 | 0.0 | 62.0 | 38.0 | 2.37 |
| 28061 | Tannin | | | | | | | | | | |
| | Cycle-2 | | | | | | | | | | |
| CW | FD 8 | 50.5 | 17.3 | 16.6 | 5.8 | 7.2 | 2.6 | 0.0 | 84.4 | 15.6 | 1.34 |
| 98117 | Tannin | | | | | | | | | | |
| | Cycle-0 | | | | | | | | | | |
| CW | FD 8 | 45.4 | 20.5 | 15.6 | 10.5 | 5.5 | 2.5 | 0.0 | 81.5 | 18.5 | 1.41 |
| 88122 | Tannin | | | | | | | | | | |
| | Cycle-0 | | | | | | | | | | |

| | | | | | | | | | | |
|---|---|---|---|---|---|---|---|---|---|---|
| AVERAGE OF 2 | 47.9 | 18.9 | 16.1 | 8.1 | 6.4 | 2.6 | 0.0 | 82.9 | 17.1 | 1.37 |
| Dormancy 8 | | | | | | | | | | |
| Cycle-0 entries | | | | | | | | | | |
| =Grnd MN | 33.3 | 20.0 | 20.1 | 12.5 | 12.7 | 1.4 | | 73.4 | 26.6 | 1.79 |
| =LSD | 22.4 | 15.2 | 11.4 | 17.2 | 10.2 | 3.7 | | 19.6 | 19.6 | 0.62 |
| =CV | 44.7 | 50.3 | 37.6 | 91.2 | 53.1 | 170.9 | | 17.7 | 48.8 | 22.9 |
| =F trt | 5.1 | 0.8 | 0.9 | 0.5 | 9.7 | 0.9 | | 2.6 | 2.6 | 6.2 |
| =F rep | 0.7 | 1.4 | 2.0 | 0.0 | 1.1 | 0.6 | | 0.4 | 0.4 | 0.6 |
| R2 | 63.4 | 53.8 | 61.9 | 12.1 | 75.6 | 38.3 | | 47.4 | 47.4 | 65.9 |

Note:
FD = Forage Dormancy Group.

### Example 5

### Breeding for Alfalfa Plants with Condensed Tannins: Cycle-3

Three experimental cages have been established with 3 cycles of selection for increased tannin levels. One population is a dormancy 6, Cycle-2 population that had been advanced in the greenhouse. The other two populations were ‘CW 29053’ (Cycle-2, FD 9) and ‘CW 28061’ (Cycle-2, FD 8).

All three populations were planted in the fall in 9×18 cone inserts that fit into 10-20 flats. Plants were thinned to 1 plant per cone. Plants with weak seedling vigor were culled. The plants were clipped for the first time in mid February, and sampling was initiated on March 25. About 25% of the vigorous plants were saved from the first sampling. All plants were then re-sampled 1 month later. About 60% of the plants were saved that remained at that time. The final selection intensity was about 15% of the plants that had good seedling vigor. As a result of this selection scheme, the following three varieties were produced:

‘CW 39061’—155 plants selected from ‘CW 29053’ were transplanted to make the Cycle-3 dormancy 9 tannin experimental cage.

‘CW 38069’—143 plants selected from ‘CW 28061’ were transplanted to make the Cycle-3 dormancy 8 tannin experimental cage.

‘CW 36081’—158 plants selected from a FD 6 tannin Cycle-2 greenhouse population were transplanted to make the Cycle-3 dormancy 6 tannin experimental cage.

### Example 6

### Breeding Methods

Open-Pollinated Populations. The improvement of open-pollinated populations of alfalfa depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population which is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). Detailed breeding methodologies specifically applicable to alfalfa are provided in Alfalfa and Alfalfa Improvement, supra.

Mass Selection. In mass selection, desirable individual plants are chosen, harvested, and the seed composited without progeny testing to produce the following generation. Since selection is based on the maternal parent only, and their is no control over pollination, mass selection amounts to a form of random mating with selection. As stated above, the purpose of mass selection is to increase the proportion of superior genotypes in the population.

### Example 7

### Synthetic Alfalfa Varieties

A synthetic variety is produced by crossing a number of selected genotypes, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-300 being the average. Broad based synthetics formed from 100 or more clones would be expected to be more stable during seed multiplication than narrow based synthetics.

Synthetics in alfalfa are used in advanced generations as commercial cultivars. The parents are always selected for some particular trait or traits but seldom for combining ability per se. Synthetic cultivars permit the expression of heterosis to a degree, usually less than hybrids, while providing a practical method for seed multiplication.

Parents for synthetic cultivars in alfalfa are selected by many different methods. In an open breeding system the parents can be selected from such diverse sources as ecotypes, cultivars, and experimental strains. Although production of a synthetic cultivar is relatively simple, a wise choice of parents for the Syn 0 generation is crucial, for this will determine the performance of the synthetic. Decisions as to which and how many parents to include, fix the minimum degree of inbreeding that the eventual cultivar will sustain in subsequent generations.

The plants with the highest scores within each dormancy/crossing group were placed in breeders seed cages and allowed to cross pollinate freely by using bees. Typically the leaf cutter bee (Megachile sp.) is used for pollination in cages but it should be noted that honey bees (Apis mellifera L.), alkali bees (Nomia melanderi) and common feral bees all effectively pollinate alfalfa. Cycle-2 seed will be harvested and planted to produce Cycle-2 plants which will be screened. The results will be compared to those obtained by screening the Cycle-0 and Cycle-1 plants. The results will show that the Cycle-2 plants have a higher average staining score than either the Cycle-0 or Cycle-1 plants.

### Example 8

### Transgenic Alfalfa with Detectable Tannin Levels

One of skill in the art would recognize that the alfalfa plants of the instant invention need not be produced solely by using classical plant breeding methodology. Recombinant DNA techniques allow plant researchers to circumvent the limitations of conventional plant breeding by enabling plant geneticists to identify and clone specific genes for desirable traits. Once the foreign genes have been introduced into a plant, that plant can than be used in conventional plant breeding schemes (e.g., pedigree breeding, single-seed-descent breeding schemes, reciprocal recurrent selection, mass selection, progeny test selection, clonal breeding) to produce progeny which also contain the gene of interest.

Standard techniques well known to those skilled in the art can be used to identify, locate and isolate the genes associated with the increased tannin levels obtained in the present invention. Furthermore, the promoters and modifying sequences associated with such genes can also be identified, located and isolated using the same techniques. The isolated nucleic acids can be used to produce transgenic cells, tissues and whole organisms, especially transgenic plant cells, plant tissues and whole plants.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in U.S. Pat. Nos. 5,451,513, 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation (see, e.g., U.S. Pat. Nos. 5,405,765, 5,472,869, 5,538,877, 5,538,880, 5,550,318, 5,641,664, 5,736,369 and 5,736369; Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); and, Raineri et al., Bio/Tech. 8:33-38 (1990)).

Transgenic alfalfa plants have been produced by many of these methods including, but not limited to, agrobacterium-mediated transformation (Wang et al., Australian Journal of Plant Physiology 23(3):265-270 (1996); Hoffman et al., Molecular Plant-Microbe Interactions 10(3):307-315 (1997); Trieu et al., Plant Cell Reports 16:6-11 (1996)) and particle acceleration (U.S. Pat. No. 5,324,646).

### Example 9

### Cell and Tissue Culture of Alfalfa

Further reproduction of the alfalfa varieties of the present invention can occur by cell and tissue culture and regeneration. Thus, another aspect of this invention is to provide cells which upon growth and differentiation produce alfalfa plants which have detectable levels of tannins, including condensed tannins. Yet another embodiment is a tissue culture of regenerable cells, where the cells include genetic material that convey the ability to produce detectable tannins, including condensed tannins. Some embodiments include such a tissue culture that includes cultured cells derived, in whole or in part, from a plant part selected from the group consisting of leaves, roots, root tips, root hairs, anthers, pistils, stamens, pollen, ovules, flowers, seeds, embryos, stems, buds, cotyledons, hypocotyls, cells and protoplasts.

In one embodiment, this invention provides cells which upon growth and differentiation produce alfalfa plants having all or substantially all of the physiological and morphological characteristics of alfalfa varieties ‘CW 28061’ and/or ‘CW 29053’.

Methods of producing alfalfa plants from tissue culture are well known by the ordinary artisan. See, for example, Daniel C. W. Brown, HortScience 23(3):526-531 (1988); Bingham, E. T., Crop Science 15:719-721 (1975); Fuentes et al., Plant Cell, Tissue and Organ Culture 34:299-302(1993); Hanson et al., Crop Science 27:1084 (1987); Ray et al., Crop Science 29:1545-1548 (1989); Seitz et al., In Vitro Cellular & Developmental Biology 24:1047-1052 (1988); Bingham et al., Alfalfa Tissue Culture, pages 903-929, In Alfalfa and Alfalfa Improvement, Hanson et al. (ed.), American Society of Agronomy, Monograph No. 29 (1988); and U.S. Pat. Nos. 5,324,646; 5,731,202; 5,908,974; 5,994,626; 6,127,599; 6,143,951; 6,359,195; 6,563,019 and 6,566,137, each of which is incorporated herein in their entirety.

Initiation of callus from immature anthers, immature ovaries, cotyledons, internode sections, and seedling hypocotyls of ‘CW 28061’ and/or ‘CW 29053’ can be achieved on Blaydes medium supplemented with various combinations and concentrations of kinetin (K), ∀-naphthalene acetic acid (NAA), and 2,4-dichlorophenoxyacetic acid (2,4-D). See, for example, Saunders, J. W. and E. T. Bingham, Crop Science 12(6):804-808 (1972). Whole alfalfa plants can be produced from the callus tissue, wherein the alfalfa plants have the same or substantially the same morphological and physiological characteristics as the plant from which the calli were derived.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. An alfalfa variety, wherein about 25% of plants of said variety score 3.0 or higher for leaf tannins using the DMACA-HCL protocol when grown in field conditions with day temperatures reaching between about 90 and about 100 degrees F. and night temperatures between about 60 and about 70 degrees F.

2. An alfalfa variety, wherein said variety has been produced by selection of plants for increased leaf tannins when grown in conditions selected from field and greenhouse conditions, said selection comprising a selection intensity of approximately 15% over at least one generation.

3. An alfalfa variety, wherein said variety confers improved bloat safety when used as feed for ruminants as compared to using the Weston variety as feed for ruminants.

4. Seed of the alfalfa variety of claim 1, claim 2 or claim 3.

5. Pollen of the alfalfa variety of claim 1, claim 2 or claim 3.
